(19) United States
(12) Patent Application Publication
(10) Pub. No.: US 2019/0321327 A1
(43) Pub. Date: Oct. 24, 2019

(54) METHOD OF TREATING HYPERGLYCEMIA

(71) Applicant: Center Laboratories, Inc., Taipei City (TW)

(72) Inventors: Jui-Pao HSU, Taipei City (TW); Guang-Tzuu SHANE, Taipei City (TW); Meng-Ju LEE, Taipei City (TW); Yi-Ping LIAO, Taipei City (TW); Yu-Yin YEH, Taipei City (TW); Shu-Hsien CHANG, Taipei City (TW)

(73) Assignee: Center Laboratories, Inc., Taipei City (TW)

(21) Appl. No.: 16/364,121

(22) Filed: Mar. 25, 2019

Related U.S. Application Data
(63) Continuation-in-part of application No. 15/957,956, filed on Apr. 20, 2018, now Pat. No. 10,278,943.

(51) Int. Cl. A61K 31/277; A61K 38/28; A61P 3/10

(52) U.S. Cl. CPC A61K 31/277; A61P 3/10; A61K 38/28

(57) ABSTRACT

Disclosed herein are methods for treating conditions and/or disorders related to hyperglycemia. In particular, the present invention relates to methods of using (R)-(+)-verapamil or a pharmaceutically acceptable salt thereof and insulin or an insulin analogue in the treatment of mellitus and/or disorders related to diabetes mellitus.

### CROSS-REFERENCES TO RELATED APPLICATIONS

This application is a continuation-in-part application of U.S. application Ser. No. 15/957,956 filed Apr. 20, 2018, which is a continuation application of U.S. application Ser. No. 15/597,200 filed May 17, 2017, which claims the benefit of U.S. Provisional Application Ser. No. 62/339,131 filed May 20, 2016; the contents of the related applications are incorporated herein by reference.

### BACKGROUND OF THE INVENTION

### 1. Field of the Invention

The present disclosure is generally directed to methods of treating hyperglycemia, such as diabetes mellitus, with a combination of (R)-(+)-verapamil or a pharmaceutically acceptable salt thereof, and insulin or an analogue thereof.

### 2. Description of Related Art

Diabetes mellitus is a condition in which a person's body does not produce enough, or does not properly respond to, insulin. Insulin is a hormone produced in the pancreas that enables cells to absorb glucose to turn it into energy. When insulin production is insufficient or when the body does not properly respond to insulin, glucose accumulates in the blood, which can lead to various complications. While there are several forms of diabetes, three forms are the most recognized: type I diabetes, type II diabetes, and gestational diabetes. Additionally, prediabetes is recognized as preceding diabetes and exists when blood glucose levels that are higher than normal but not yet high enough to be diagnosed as diabetes.

Type I diabetes or insulin-dependent diabetes mellitus (IDDM) is a metabolic disorder caused by destruction of the insulin-producing beta cells in the pancreas, which leads to insulin deficiency and high levels of glucose in plasma. The onset of type I diabetes generally results from an autoimmune etiology; however, idiopathic causes of beta cell destruction can occur for type I. Type 1 diabetes can affect children or adults, but was traditionally termed “juvenile diabetes” because it represents a majority of the diabetes cases in children.

Type II diabetes or non-insulin-dependent diabetes mellitus (NIDDM) has been found to possess inheritable aspects which can be greatly impacted by external environmental factors. The underlying etiologies of type II diabetes include deficiencies in insulin-producing beta cells; altered response to insulin by muscle, adipose, and liver cells; and abnormalities in the regulating mechanisms responsible for controlling carbohydrate and lipid metabolism following ingestion of food. Modulation in insulin-sensitivity is affected by environmental factors and behaviors, mostly a sedentary lifestyle and obesity. The cellular mechanisms that contribute to modulation of muscle and adipose cell sensitivity to insulin are complex and are not well understood. It is believed that altering insulin signaling pathways, increasing the amount of intracellular fat, and elevating levels of free fatty acids and other adipose tissue products can impact insulin-sensitivity.

Gestational diabetes occurs in pregnant women who have not previously been diagnosed with diabetes but who have high glucose levels during pregnancy. Gestational diabetes affects about 4% of all pregnant women and may precede development of type II diabetes.

If not properly controlled or stabilized, a hyperglycemic state has been associated with comorbidities including cardiovascular disease, vision impairment, various forms of neuropathy and cognitive impairment, stroke, and peripheral vascular disease. The common therapeutic approach, in addition to major modifications in an individual's dietary nutrition and physical activity, includes the use of anti-hyperglycemic drugs and insulin. Since the disease is chronic and progressive, and so far no treatment is able to reverse the progression, and thus there remains in this field a need of an improved medicament for treating conditions, diseases and/or disorders associated with hyperglycemia.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

The present invention relates to a medicament, which effectively reduces the HbA_{1c} level of a hyperglycemia subject. The present invention therefore is useful for treating conditions related to hyperglycemia, which includes, but is not limited to, type I, type II diabetes mellitus, gestational diabetes, other forms of diabetes and/or disorders related thereto.

Accordingly, one aspect of the present disclosure relates to the combined use of (R)-(+)-verapamil or a pharmaceutically acceptable salt thereof and insulin or an insulin analogue for the manufacture of a pharmaceutical composition for the treatment of diabetes mellitus and/or disorders related to diabetes mellitus.

The pharmaceutical composition of the present disclosure comprises (R)-(+)-verapamil or a pharmaceutically acceptable salt thereof, insulin or an insulin analogue, and a pharmaceutically acceptable excipient.

According to preferred embodiments of the present disclosure, the (R)-(+)-verapamil or a pharmaceutically acceptable salt thereof comprised in the pharmaceutical composition is in the form of a crystalline.

According to preferred embodiments of the present disclosure, the (R)-(+)-verapamil comprised in the pharmaceutical composition is in the form of hydrochloride salt.

Suitable examples of the insulin analogue for use in the pharmaceutical composition include, but are not limited to, glargine, degludec and detemir. In one preferred embodiment, the insulin analogue comprised in the pharmaceutical composition is glargine.

According to preferred embodiments of the present disclosure, the pharmaceutical composition comprising (R)-(+)-verapamil or a pharmaceutically acceptable salt thereof, and the insulin or its analogue are independently suitable for oral, intravenous, intramuscular, intraperitoneal, subcutaneous, or transmucosal administration.

Another aspect of the present invention relates to a method of treating hyperglycemia, particularly, diabetes mellitus and/or disorders related to diabetes mellitus. The method includes respectively administering to a subject suffering from diabetes mellitus and/or disorders related to diabetes mellitus effective amounts of (R)-(+)-verapamil or a pharmaceutically acceptable salt thereof and insulin or an insulin analogue to alleviate or ameliorate the symptoms associated with diabetes mellitus and/or disorders related to diabetes mellitus.

Suitable examples of insulin analogue for use in the present method include, but are not limited to, glargine, degludec and detemir. In one preferred embodiment, the insulin analogue suitable for use in the present method is glargine.

According to preferred embodiments of the present disclosure, the (R)-(+)-verapamil or a pharmaceutically acceptable salt thereof is administered in the form of a crystalline.

According to preferred embodiments of the present disclosure, the (R)-(+)-verapamil is administered in the form of a hydrochloride salt.

According to embodiments of the present disclosure, the (R)-(+)-verapamil or a pharmaceutically acceptable salt thereof is administered in the amount from about 15 to 1,000 mg/day. Preferably, the (R)-(+)-verapamil or a pharmaceutically acceptable salt thereof is administered in the amount from about 25 to 800 mg/day. More preferably, the (R)-(+)-verapamil or a pharmaceutically acceptable salt thereof is administered in the amount from about 30 to 600 mg/day.

According to embodiments of the present disclosure, the insulin or the insulin analogue is administered in the amount from about 0.05 to 3 units/kg/day. Preferably, the insulin or the insulin analogue is administered in the amount from about 0.1 to 2 units/kg/day. More preferably, the insulin or the insulin analogue is administered in the amount from about 0.2 to 1 units/kg/day.

According to embodiments of the present disclosure, the (R)-(+)-verapamil or a pharmaceutically acceptable salt thereof and the insulin or its analogue may be respectively administered orally, intravenously, intramuscularly, intraperitoneally, subcutaneously, or transmucosally.

Many of the attendant features and advantages of the present disclosure will becomes better understood with reference to the following detailed description considered in connection with the accompanying drawings.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### 1. Definitions

For convenience, certain terms employed in the specification, examples and appended claims are collected here. Unless defined otherwise, all technical and scientific terms used herein have the same meaning as commonly understood by one of the ordinary skill in the art to which this invention belongs.

The singular forms “a”, “and”, and “the” are used herein to include plural referents unless the context clearly dictates otherwise. The term “about” as used herein generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, or reflection angles disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The term “diabetes mellitus” refers to type I, type II diabetes mellitus, gestational diabetes, and other forms of diabetes. Other forms diabetes may be latent autoimmune diabetes of adult (LADA), congenital diabetes, steroid diabetes, pancreatic defects related diabetes (e.g., chronic pancreatitis related diabetes, cystic fibrosis related diabetes, pancreatic neoplasia related diabetes, hemochromatosis related diabetes, and fibrocalculous pancreatopathy related diabetes), endocrinopathy related diabetes (e.g., acromegaly related diabetes, Crushing syndrome related diabetes, hyperthyroidism related diabetes, pheochromocytoma related diabetes, and glucagonoma related diabetes), infection related diabetes (e.g., cytomegalovirus infection related diabetes, and coxackievirus B related diabetes), diabetic angiopathy (e.g., diabetic retinopathy and diabetic nephropathy), and drug related diabetes (e.g., glucocorticoids related diabetes, thyroid hormone related diabetes, β-adrenergic agonists related diabetes, and statins related diabetes). Frequently correlated with type II diabetes mellitus are one or more of the metabolic syndrome, obesity, insulin resistance, dyslipidemia and a pathological glucose tolerance. Subjects with diabetes mellitus manifest varying degrees of increased blood pressure, increased levels of cholesterol and/or triglycerides, increased levels of uric acid and increased levels of factors that promote coagulation. Therefore, “disorders related to diabetes mellitus” as used herein refers to hypertension, hyperlipidemia, hyperuricemia, gout and hypercoagulability, i.e. an abnormal, increased tendency to form clots inside blood vessels. These disorders are well-recognized risk factors for atherosclerotic macrovascular as well as microvascular diseases. Atherosclerotic macrovascular diseases include myocardial infarction, stroke and limb amputation. Microvascular complications involve blindness, renal diseases and debilitating neuropathies.

The term “treatment” as used herein are intended to mean obtaining a desired pharmacological and/or physiologic effect, e.g., reducing blood glucose or HbA1c level in a hyperglycemia subject. The effect may be prophylactic in terms of completely or partially preventing a disease or symptom thereof and/or therapeutic in terms of a partial or complete cure for a disease and/or adverse effect attributable to the disease. “Treatment” as used herein includes, but is not limited to, preventative (e.g., prophylactic), curative or palliative treatment of a disease in a mammal, particularly human; and includes: (1) preventative (e.g., prophylactic), curative or palliative treatment of a disease or condition (e.g., diabetes mellitus or disorders related thereto) from occurring in an individual who may be pre-disposed to the disease but has not yet been diagnosed as having it; (2) inhibiting a disease (e.g., by promoting the proliferation of insulin-producing beta cells or suppressing apoptosis of these cells); or (3) relieving a disease (e.g., reducing symptoms associated with the disease).

The term “administered”, “administering” or “administration” are used interchangeably herein to refer a mode of delivery, including, without limitation, orally, intraveneously, intramuscularly, intraperitoneally, intraarterially, transmucosally (e.g., inhalation, and intranasally), or subcutaneously administering of an agent (e.g., (R)-(+)-verapamil and insulin or its analogue) of the present invention.

The term “an effective amount” as used herein refers to an amount effective, at dosages, and for periods of time necessary, to achieve the desired result with respect to the treatment of a disease resulted from hyperglycemia. For example, in the treatment of diabetes mellitus, an agent (i.e., (R)-(+)-verapamil and insulin or its analogue) which decrease, prevents, delays or suppresses or arrests any symptoms related to diabetes mellitus would be effective. An effective amount of an agent is not required to cure a disease or condition but will provide a treatment for a disease or condition such that the onset of the disease or condition is delayed, hindered or prevented, or the disease or condition symptoms are ameliorated. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the like. Effective amount may be expressed, for example, as the total mass of the active agent (e.g., in grams, milligrams or micrograms) per day. The effective amount may be divided into one, two or more doses in a suitable form to be administered at one, two or more times throughout a designated time period.

The term “subject” or “patient” is used interchangeably herein and is intended to mean a mammal including the human species that is treatable by the compound of the present invention. The term “mammal” refers to all members of the class Mammalia, including humans, primates, domestic and farm animals, such as rabbit, pig, sheep, and cattle; as well as zoo, sports or pet animals; and rodents, such as mouse and rat. Further, the term “subject” or “patient” intended to refer to both the male and female gender unless one gender is specifically indicated. Accordingly, the term “subject” or “patient” comprises any mammal which may benefit from the treatment method of the present disclosure. Examples of a “subject” or “patient” include, but are not limited to, a human, rat, mouse, guinea pig, monkey, pig, goat, cow, horse, dog, cat, bird and fowl. In a preferred embodiment, the subject is a human.

The term “excipient” as used herein means any inert substance (such as a powder or liquid) that forms a vehicle/carrier for the active agent. The excipient is generally safe, non-toxic, and in a broad sense, may also include any known substance in the pharmaceutical industry useful for preparing pharmaceutical compositions such as, fillers, diluents, agglutinants, binders, lubricating agents, glidants, stabilizer, colorants, wetting agents, disintegrants, and etc.

The term “racemic” as used herein refers to a mixture of the (R)- and (S)-enantiomers, or stereoisomers, of verapamil, in which neither enantiomer or stereoisomer is substantially purified from the other.

### II. Treatment of Diabetes Mellitus and Disorders Related Thereto

2.1 Treatment Methods

Verapamil (e.g., 2-(3,4-dimethoxyphenyl)-5-[2-(3,4-dimethoxyphenyl)ethyl-methylamino]-2-propan-2-ylpentanenitrile) is a known drug with various medicinal indications. Traditionally, it is used for treating coronary disease, such as hypertension. The compound has a stereogenic center, hence can be separated into its optical enantiomers. The (S)-enantiomer is known to possess the majority of the calcium channel antagonist activity, whereas the (R)-enantiomer is known to possess agonist activity toward somatostatin receptor 2, and antagonist activity toward orexin receptors 1 and 2, dopamine D2L receptor, sodium and calcium channels (see WO 2011/057471A1); accordingly, the (R)-enantiomer is useful as a medicament for treating diseases or conditions related to these receptors in a human subject.

The present invention in general, relates to the combinational use of (R)-(+)-verapamil and insulin or its analogue, in which the combination is capable of reducing the increasing level of HbA_{1c} in a diabetic subject. Accordingly, the (R)-(+)-verapamil and the insulin or the insulin analogue, may be manufactured into a medicament for use in the treatment of diabetes mellitus and/or disorders related thereto.

In this regard, a particular aspect of the present invention relates to a method of treating a subject suffering from diabetes mellitus, particularly, diabetes mellitus and/or disorders related to diabetes mellitus. The method includes the step of, administering to the subject an effective amount of (R)-(+)-verapamil or a pharmaceutically acceptable salt thereof (i.e., (R)-(+)-verapamil HCl) and insulin or an insulin analogue.

(R)-(+)-verapamil may be obtained from racemic mixture of verapamil by high performance liquid chromatography (HPLC) separation or resolution of the enantiomers using any available means, such as optically active resolving acid. Alternatively, (R)-(+)-verapamil may be synthesized by stereospecific synthesis using any method known in the related art. Stereospecific synthesis in general can result in products with high enantiomeric purity. In cases when the enantiomeric purity is not sufficient, then the synthetic product may be subject to further purification process to enhance the enantiomeric purity by separating (R)-(+)-verapamil from (S)-(−)-verapamil. Examples of processes for resolving racemic verapamil to produce (R)-(+)-verapamil are well known to those of ordinary skill in the art.

According to some preferred embodiments, the (R)-(+)-verapamil or a pharmaceutically acceptable salt thereof is administered in a crystalline form. The crystalline of (R)-(+)-verapamil or a pharmaceutically acceptable salt thereof may be produced by any known crystallization method, e.g., saturation method. In one example, (R)-(+)-verapamil HCl is dissolved in suitable solvent(s), which include but are not limited to, ethyl acetate, toluene, and 1,4-dioxane/heptane (1:1), until a saturated solution is obtained; the saturated solution is then cooled to form (R)-(+)-verapamil HCl crystals therefrom.

According to preferred embodiments, the (R)-(+)-verapamil suitable for use in the present invention is in the form of hydrochloride salt, that is, (R)-(+)-verapamil HCl.

According to preferred embodiments, the (R)-(+)-verapamil or a pharmaceutically acceptable salt thereof is administered with insulin or an insulin analogue to ameliorate or alleviate the hyperglycemia state of the subject.

Suitable examples of the insulin analogue include, but are not limited to, glargine, degludec, and detemir.

According to preferred embodiments of the present disclosure, (R)-(+)-verapamil HCl and glargine are respectively administered to the subject.

According to embodiments of the present disclosure, (R)-(+)-verapamil or a pharmaceutically acceptable salt thereof may be administered to the subject in need of such treatment in the amount of 15 to 1,000 mg/day, such as 15, 20, 25, 30, 35, 40, 50, 60, 70, 75, 80, 90, 100, 110, 120, 125, 130, 140, 150, 160, 170, 175, 180, 190, 200, 210, 220, 230, 240, 250, 260, 270, 280, 290, 300, 310, 320, 330, 340, 350, 360, 370, 375, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1,000 mg/day; preferably, the (R)-(+)-verapamil or a pharmaceutically acceptable salt thereof is administered in the amount from about 25 to 800 mg/day, such as 25, 30, 35, 40, 50, 60, 70, 75, 80, 90, 100, 110, 120, 125, 130, 140, 150, 160, 170, 175, 180, 190, 200, 210, 220, 230, 240, 250, 260, 270, 280, 290, 300, 310, 320, 330, 340, 350, 360, 370, 375, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 mg/day; more preferably, the (R)-(+)-verapamil or a pharmaceutically acceptable salt thereof is administered in the amount from about 30 to 600 mg/day, such as 30, 35, 40, 50, 60, 70, 75, 80, 90, 100, 110, 120, 125, 130, 140, 150, 160, 170, 175, 180, 190, 200, 210, 220, 230, 240, 250, 260, 270, 280, 290, 300, 310, 320, 330, 340, 350, 360, 370, 375, 380, 390, 400, 410, 420, 430, 440, 450, 460, 470, 480, 490, 500, 510, 520, 530, 540, 550, 560, 570, 580, 590, 600 mg/day.

According to embodiments of the present disclosure, the insulin or the insulin analogue may be administered to the subject in need of such treatment in the amount of 0.05 to 3 units/kg/day, such as 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, and 3.0 units/kg/day; preferably, the insulin or the insulin analogue is administered to the subject in need of such treatment in the amount of 0.1 to 2 units/kg/day, such as 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, and 2.0 units/kg/day; more preferably, the insulin or the insulin analogue is administered to the subject in need of such treatment in the amount of 0.2 to 1 units/kg/day, such as 0.2, 0.3, 0.4, 0.5, 0.6, 0.7, 0.8, 0.9 and 1.0 units/kg/day.

According to preferred embodiments, (R)-(+)-verapamil or a pharmaceutically acceptable salt thereof is administered to a subject in need thereof along with glargine, in which the combined treatment results in the reduction in the level of HbA_{1c}.

According to embodiments of the present disclosure, the (R)-(+)-verapamil or a pharmaceutically acceptable salt thereof, as well as the insulin or the insulin analogue, may be respectively administered via any suitable route, which includes, but is not limited to, oral, intravenous, intramuscular, intraperitoneal, intraarterial, transmucosal, and subcutaneous route. In preferred embodiment, (R)-(+)-verapamil hydrochloride is orally administered to the subject in need thereof; while the insulin or the insulin analogue (e.g., glargine) is subcutaneously injected into the subject in need thereof.

2.3 Pharmaceutical Composition

A further aspect of the present invention relates to pharmaceutical compositions for the treatment of diabetes mellitus, particularly diabetes mellitus and/or disorders related to diabetes mellitus.

To produce the pharmaceutical composition, the active components of the present disclosure, (R)-(+)-verapamil or a pharmaceutically acceptable salt thereof (preferably, a crystalline of (R)-(+)-verapamil), and insulin or its analogue, are mixed with suitable excipients and formulated into a dosage form suitable for administering orally, intraveneously, intramuscularly, intraperitoneally, intraarterially, intracranially, transmucosally (e.g., inhalation, buccal, and intranasally), or subcutaneously. Examples of the insulin analogue include, but are not limited to, glargine, degludec and detemir. Suitable excipients are known to those of skill in the art and described, for example, in Handbook of Pharmaceutical Excipients (Kibbe (ed.), 3^{rd} Edition (2000), American Pharmaceutical Association, Washington, D.C.), and Remington's Pharmaceutical Sciences (Gennaro (ed.), 20^{th} edition (2000), Mack Publishing Inc., Easton, Pa.), which for their disclosure relating to excipients and dosage forms, are incorporated herein by reference. For example, suitable excipients include, but are not limited to, starches, sugars, microcrystalline cellulose, diluents, granulating agents, lubricants, binders, disintegrating agents, wetting agents, lubricants, emulsifiers, coloring agent, release agents, coating agents, sweetening agents, flavoring agents, preservatives, plasticizers, gelling agents, thickeners, hardeners, setting agents, suspending agents, surfactants, humectants, carriers, stabilizers, antioxidants, and combinations thereof.

The pharmaceutical composition are typically provided in dosage forms suitable for administration to a subject by any desired route. One of skill in the art is familiar with various dosage forms that are suitable for use in the present invention. The most suitable route in any given case will depend on the nature and severity of the disease being treated and/or managed. For example, the pharmaceutical compositions may be formulated for administration orally, intraveneously, intramuscularly, intraperitoneally, intraarterially, intracranially, transmucosally (e.g., inhalation, buccal, and intranasally), or subcutaneously. In some embodiments, the pharmaceutical composition is administered orally. In other embodiments, the pharmaceutical composition is administered intraveneously.

The dosage form of the pharmaceutical composition suitable for oral administration includes, for example, tablets, pills, granules, powders, solutions, suspensions, syrups or capsules. As a method of producing solid dosage form such as a tablet, a pill, granule or powder, it can be formed by conventional techniques using a pharmaceutically acceptable carrier such as excipient, binder, or disintegrant and etc. The solid dosage form for oral administration may optionally be scored or prepared with coating and shells, such as entering coatings, and coatings for modifying the rate of release. Further, any of the solid dosage form may be encapsulated in soft and hard gelatin capsules using any of the excipients known in the art.

The active components, such as (R)-(+)-verapamil or a pharmaceutically acceptable salt thereof and insulin or its analogue, may also be formulated into a liquid dosage form for oral administration. Suitable formulation include emulsion, solutions, suspension or syrup, it can be produced by conventional techniques using diluents commonly used in the art, such as water, glycerol esters, alcohols, vegetable oils, and etc. The liquid formulation may optionally include adjuvants such as wetting agents, emulsifying agents, and suspending agents, sweetening, flavoring, coloring, and preservative agents. The liquid formulation may also be filled into soft gelatin capsules. For example, the liquid may include a solution, suspension, emulsion, precipitate, or any other desired liquid media carrying the (R)-(+)-verapamil and insulin. The liquid may be designed to improve the solubility of the (R)-(+)-verapamil and insulin upon release, or may be designed to form a drug-containing emulsion or dispersed phase upon release. Examples of such techniques are well known in the related art. Soft gelatin capsules may be coated, as desired, with a functional coating, such as to delay the release of the drug.

In the case of parenteral administration, active components (i.e., (R)-(+)-verapamil or a pharmaceutically acceptable salt thereof and insulin or its analogue) may be formulated into injectable forms for intravenous, subcutaneous or intramuscular administration. An injection can be prepared by dissolving the active components of the present disclosure in water soluble solution such as physiological saline, or water insoluble solution consisting of organic esters such as propylene glycol, polyethylene glycol, or vegetable oils (e.g., sesame oil).

In the case of transdermal administration, for example, a dosage form as an ointment or a cream can be employed. The ointment can be produced by mixing the active components of the present disclosure with fats or oils and etc; and the cream can be produced by mixing the active components of the present disclosure with emulsifiers. The transdermal formulation may be a liquid or a powdery formulation. In a liquid formulation water, salt solution, phosphate buffer, acetate buffer and etc may be used as a base; it may also contain surfactants, antioxidants, stabilizers, preservatives or tackifiers. In a powdery formulation, it may contain water-absorbing materials such as water-soluble polyacrylates, cellulose low-alkyl esters, polyethylene glycol polyvinyl pyrrolidone, amylase and etc, and non-water absorbing materials such as cellulose, starches, gums, vegetable oils or cross-linked polymers. Further, antioxidants, colorants, preservatives may be added to the powdery formulation. The liquid or powdery formulation may be administered by use of a spray apparatus.

In the case of rectal administration, it may be in the form of suppository using a gelatin soft capsule.

In case of inhalation through nose or mouth, a solution or suspension containing the active components of the present disclosure and a pharmaceutical excipient generally accepted for this purpose is inhaled through an inhalant aerosol spray. Alternatively, the active components of the present disclosure in the form of a powder may be administered through inhalator that allows direct contact of the powder with the lung. To these formulations, if necessary, pharmaceutical acceptable carriers such as isotonic agents, preservatives, dispersions, or stabilizers may be added. Further, if necessary, these formulations may be sterilized by filtration, or by treatment with heat or irradiation.

The effective amount of the active compounds of the present disclosure (i.e., (R)-(+)-verapamil or a pharmaceutically acceptable salt thereof; and insulin or its analogue) suitable for treating diabetes mellitus and/or disorders related thereto varies with the route of administration, or condition, age, sex, or weight of the subject receiving the treatment.

According to embodiments of the present disclosure, the present pharmaceutical composition comprises 10 to 1,000 mg of (R)-(+)-verapamil or a pharmaceutically acceptable salt thereof; 3 to 180 units of insulin or its analogue; and a pharmaceutically acceptable excipient. More preferably, the composition comprises 25 to 800 mg of (R)-(+)-verapamil or a pharmaceutically acceptable salt thereof; 6 to 120 units of insulin or its analogue; and a pharmaceutically acceptable excipient. Most preferably, the present pharmaceutical composition comprises 30 to 600 mg of (R)-(+)-verapamil or a pharmaceutically acceptable salt thereof; 12 to 60 units of insulin or its analogue; and a pharmaceutically acceptable excipient. In general, the pharmaceutical composition is administered to the subject in single or divided doses 2, 3, 4 or more times each day. Alternatively, the dose may be delivered once every 2, 3, 4, 5 or more days. In one preferred embodiment, the pharmaceutical composition is administered once per day. In another embodiment, the pharmaceutical composition is administered twice per day.

2.3 Kits

Additionally or optionally, (R)-(+)-verapamil or a pharmaceutically acceptable salt thereof and insulin or its analogue may be provided in a separate formulation and co-administered to the subject with the present pharmaceutical composition. Such separate formulations may be administered independently or concurrently via the same or different route. Accordingly, a further aspect of the present invention relates to kits for the treatment of diabetes mellitus, particularly diabetes mellitus and/or disorders related to diabetes mellitus.

Accordingly, embodiments of the present disclosure aim at providing a kit, which comprises a first container containing therein (R)-(+)-verapamil or a pharmaceutically acceptable salt thereof; a second container containing therein insulin or an insulin analogue; and a legend associated with the kit for directing a user how to use the kit. The legend may be in a form of label, package insert, pamphlet, tape, CD, VCD or DVD.

According to preferred embodiments of the present disclosure, the (R)-(+)-verapamil or a pharmaceutically acceptable salt thereof comprised in the kit is in the form of a crystalline.

According to preferred embodiments of the present disclosure, the (R)-(+)-verapamil comprised in the kit is in the form of hydrochloride salt.

Suitable examples of the insulin analogue for use in the kit include, but are not limited to, glargine, degludec or detemir. In one preferred embodiment, the insulin analogue comprised in the kit is glargine.

According to embodiments of the present disclosure, (R)-(+)-verapamil or a pharmaceutically acceptable salt thereof and the insulin or its analogue are independently formulated into a dosage form suitable for administering orally, intraveneously, intramuscularly, intraperitoneally, intraarterially, transmucosally (e.g., inhalation, buccal, and intranasally), or subcutaneously by mixing suitable amounts of the active ingredients (e.g., (R)-(+)-verapamil or a pharmaceutically acceptable salt thereof, or the insulin) with suitable excipients. Suitable excipients are as described above.

According to some preferred embodiments of the present disclosure, one of the dosage forms in the kit is in the form of a tablet, which comprises 10 to 1,000 mg of (R)-(+)-verapamil or a pharmaceutically acceptable salt thereof, such as 10, 15, 20, 25, 30, 35, 40, 45, 50, 55, 60, 65, 70, 75, 80, 85, 90, 95, 100, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, and 1,000 mg (R)-(+)-verapamil or a pharmaceutically acceptable salt thereof, and a pharmaceutically acceptable carrier; preferably, the tablet comprises 25 to 800 mg of (R)-(+)-verapamil or a pharmaceutically acceptable salt thereof, such as 25, 30, 35, 40, 45, 50, 55, 60, 65, 70, 75, 80, 85, 90, 95, 100, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 610, 620, 630, 640, 650, 660, 670, 680, 690, 700, 710, 720, 730, 740, 750, 760, 770, 780, 790, and 800 mg (R)-(+)-verapamil or a pharmaceutically acceptable salt thereof, and a pharmaceutically acceptable carrier. More preferably, the tablet comprises 30 to 600 mg of (R)-(+)-verapamil or a pharmaceutically acceptable salt thereof, such as 30, 35, 40, 45, 50, 55, 60, 65, 70, 75, 80, 85, 90, 95, 100, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600 mg (R)-(+)-verapamil or a pharmaceutically acceptable salt thereof, and a pharmaceutically acceptable carrier.

The other dosage form in the kit is in the form of a solution, which comprises 3 to 180 units of insulin or its analogue, such as 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 and 180 units insulin or its analogue dissolved or suspended in an aqueous carrier (e.g., water); preferably, the solution comprises 6 to 120 units of insulin or its analogue, such as 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 units insulin or its analogue dissolved or suspended in an aqueous carrier; more preferably, the solution comprises 12 to 60 units insulin or its analogue, such as 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, and 60 units insulin or its analogue dissolved or suspended in an aqueous carrier.

In general, each dosages may be administered to the subject in single or divided doses 2, 3, 4 or more times each day. Alternatively, each dosages may be delivered once every 2, 3, 4, 5 or more days. In one preferred embodiment, the tablets, which independently comprises (R)-(+)-verapamil or a pharmaceutically acceptable salt thereof, are administered twice per day; while the solution that comprises the insulin is administered once per day.

The present invention will now be described in further detail with reference to the following examples. However, it should be understood that the present invention is not limited to the specified examples.

### EXAMPLES

Materials and Methods

Materials.

(R)-(+)-verapamil HCl and racemic verapamil HCl were provided by Center Laboratories, Inc. (Taipei, Taiwan, R.O.C.). Insulin glargine (LANTUS) was from Sanofi-Aventis (Germany), and HbA_{1c} assay kits were from Fujirebio (Japan).

Animals.

Non-obese diabetes (NOD) female mice (each weighted about 20-25 g) were used in the present study. NOD mice were provided by The Jackson Laboratory (Bar Harbor, Me., USA).

The mice were about 8-9 weeks old at the beginning of the experiment, and about 30-31 weeks old at the end of the experiment. They were housed in a group of 2-5 mice per cage and maintained in individually ventilated cages throughout the study in the animal facility with controlled temperature (20-26° C.), humidity (40-70%) and a 12 h/12 h light/dark cycle (light on at 7:00 a.m.) with food and water provided ad libitum. Experimental procedures for handling the mice complied with guidelines approved by the Association for Assessment and Accreditation of Laboratory Animal Care (AAALAC).

NOD Mice and Treatment

The experiment started with 190 female NOD mice, non-fasting blood glucose level in each mice was measured once a week from 8 weeks old, any reading of blood glucose>250 mg/dL was followed by a test 24 hr later. Diabetes onset was defined by two successive reading of non-fasting blood glucose>250 mg/dL. A total of 80 mice developed late onset hyperglycemia, and were thus selected for the experiment, while the rest of the mice were removed from the study.

The selected mice were dosed in accordance with the regimen provided in Table 1, in which insulin was administered once per day via subcutaneous injection (sc), while test compound (verapamil HCl in racemic form (VPM), or (R)-(+)-verapamil HCl (R-VPM)) and vehicle control were given by gavage feeding twice per day (bid, at an interval of 6 hrs), except days 0, 14, 28, 42, and 56. Animals were observed daily for their behaviors, activities, food and water intakes and etc. during the entire study.

TABLE 1
Dosing Regimen

| | | Dosage | |
|---|---|---|---|
| Group | Treatment | Insulin | Test compound |
| 1 | Vehicle | 0 U/mouse | 0 mg/kg |
| 2 | Insulin | Days 1-20: 0.2 unit/mouse | 0 mg/kg |
| | | Days 21-35: 0.5 unit/mouse | |
| | | Days 36-43: 0.8 unit/mouse | |
| | | Days 44-56: 1 unit/mouse | |
| 3 | Insulin + R-VPM | Days 1-20: 0.2 unit/mouse | 15 mg/kg |
| | | Days 21-35: 0.5 unit/mouse | |
| | | Days 36-43: 0.8 unit/mouse | |
| | | Days 44-56: 1 unit/mouse | |
| 4 | Insulin + R-VPM | Days 1-20: 0.2 unit/mouse | 30 mg/kg |
| | | Days 21-35: 0.5 unit/mouse | |
| | | Days 36-43: 0.8 unit/mouse | |
| | | Days 44-56: 1 unit/mouse | |
| 5 | Insulin + R-VPM | Days 1-20: 0.2 unit/mouse | 50 mg/kg |
| | | Days 21-35: 0.5 unit/mouse | |
| | | Days 36-43: 0.8 unit/mouse | |
| | | Days 44-56: 1 unit/mouse | |
| 6 | Insulin + VPM | Days 1-20: 0.2 unit/mouse | 30 mg/kg |
| | | Days 21-35: 0.5 unit/mouse | |
| | | Days 36-43: 0.8 unit/mouse | |
| | | Days 44-56: 1 unit/mouse | |

Body weight was measured three times weekly, water and food intake were measured twice per week. Blood samples were collected 24 hrs after the last insulin administration from tail vein and were used for the determination of blood HbA_{1c} level.

Statistics

Results were expressed as the mean±standard error of the mean (SEM). Unpaired student's t-test was used for statistical comparisons between substance-treated and vehicle-treated groups. Differences are considered significant at P<0.05.

### Example 1 Combined Treatment of (R)-(+)-Verapamil HCl and Insulin Reduced the Levels of HbA_{1c} in NOD Mice

In this example, the combined effects of insulin and (R)-(+)-verapamil HCl (R-VPM) or racemic verapamil (VPM) on HbA_{1c} was evaluated in NOD mice.

HbA_{1c} develops when haemoglobin, a protein within red blood cells that carries oxygen throughout the body joins with glucose in the blood and becomes “glycated.” Since red blood cells in a human body survive for about 120 days before renewal, thus, the level of HbA1c gives an overall picture of average blood sugar levels over such period. However, within this period of 120 days, recent glycemia has the major influence on the HbA_{1c} value, as 50% of HbA_{1c} formed in the month prior to sampling and 25% in the month before that. The level of HbA_{1c} therefore serves a good indicator for diagnosing a disease characterized in chronic hyperglycemia and a gradual progression to complications. Results are summarized in Table 2.

TABLE 2
HbA_{1c} level change in NOD mice

| | | HbA_{1c} change (%) | | | |
|---|---|---|---|---|---|
| | | weeks | | | |
| # | Treatment | 2 | 4 | 6 | 8 |
| 1 | Vehicle | 32.2 ± 9.6 | 64.1 ± 12.6 | 58.0 ± 18.6 | 70.8 ± 26.7 |
| | | | | | (NS) |
| 2 | Insulin | 4.0 ± 5.5 | 29.6 ± 9.6 | 6.1 ± 8.9 | 13.9 ± 9.7 |
| 3 | Insulin + R-VPM | 20.9 ± 5.9 | 50.4 ± 9.5 | 21.4 ± 11.4 | 34.9 ± 15.4 |
| | (15 mg/Kg) | (NS) | (NS) | (NS; | (NS) |
| | | | | p = 0.094) | |
| 4 | Insulin + R-VPM | 9.1 ± 7.4 | 22.9 ± 15.5 | −10.1 ± 9.1 | −15.3 ± 6.6 |
| | (30 mg/Kg) | (NS; | (NS; | | |
| | | p = 0.078) | p = 0.058) | | |
| 5 | Insulin + R-VPM | NS | 32.0 ± 7.1 | 17.3 ± 9.9 | NS |
| | (50 mg/Kg) | | | (NS; | |
| | | | | p = 0.053) | |
| 6 | Insulin + VPM (30 mg/Kg) | 8.2 ± 5.0 | 25.4 ± 10.6 | 3.6 ± 10.1 | NS |

NS: non-significant (p > 0.05)

In the present study, it was found that after 8 weeks of treatment, the change in HbA_{1c} levels in vehicle group were respectively 32.2%, 64.1%, 58% and 70.8% at weeks 2, 4, 6, and 8 as compared to the baseline, which was a clear indication that the test animals were in deed in hyperglycemia state. For mice treated with insulin alone, the change in HbA_{1c} level significantly reduced to about 4.0%, 29.6%, 6.1% and 13.8% respectively at weeks 2, 4, 6, and 8, a clear confirmation that insulin could effectively lower the hyperglycemia state of NOD mice. In the case when mice were treated with both insulin (0.2-1 units/kg/day) and R-VPM (at the dose of 15, 30 or 50 mg/kg), a tread in the reduction of HbA_{1c} level from weeks 2 to 8 was observed as compared to those of vehicle-treated animals. Take the group animals treated with insulin and R-VPM at the dose of 15 mg/Kg as an example, though the data points were not statistic significant as compared to the vehicle-treated animals, yet there was clearly a trend that the combined treatment of insulin and R-VPM resulted in the reduction of HbA_{1c} level. The tread was most evident for mice treated with insulin and R-VPM at 30 mg/Kg, with the changes in HbA_{1c} level independently lower than those of the insulin-treated animal (week 6: 6.1% (insulin) vs −10.1% (insulin+VPM); week 8: 13.9% (insulin) vs −15.3% (insulin+VPM)). The results suggest that the combined treatment of insulin and R-VPM could effectively ameliorate the hyperglycemia state of NOD mice, as the tendency of increasing HbA1c levels observed in vehicle-treated NOD mice were independently hampered by the combined treatment of insulin and R-verapamil.

In addition, for mice treated with the combination of insulin and VPM (30 mg/kg), there was also a tendency in reducing the increase in HbA_{1c} level at week 8 (12.7%); however, the effect was not as evident as that in R-VPM at the same dose.

Taken together the findings in this example, the combined use of insulin (0.2-1 units, qd) and (R)-(+)-verapamil (15, 30 or 50 mg/Kg, bid) improves hyperglycemia state of a test subject that reflects in the reduction of increasing HbA1c level. Accordingly, the combination of insulin and (R)-(+)-verapamil may be suitable for use as a medicament for treating diabetes mellitus and/or disorders related thereto.

It will be understood that the above description of embodiments is given by way of example only and that various modifications may be made by those with ordinary skill in the art. The above specification, examples, and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

1. A method of treating a subject having diabetes mellitus and/or disorders related to diabetes mellitus comprising administering to the subject an effective amount of (R)-(+)-verapamil or a pharmaceutically acceptable salt thereof, and insulin or an insulin analogue.

2. The method of claim 1, wherein the insulin analogue is glargine, degludec or detemir.

3. The method of claim 2, wherein the insulin analogue is glargine.

4. The method of claim 1, wherein the (R)-(+)-verapamil or a pharmaceutically acceptable salt thereof is administered to the subject in the amount of 15 to 1,000 mg/day; and the insulin or the insulin analogue is administered to the subject in the amount of 0.05 to 3 units/kg/day.

5. The method of claim 4, wherein the (R)-(+)-verapamil or a pharmaceutically acceptable salt thereof is administered to the subject in the amount of 25 to 800 mg/day; and the insulin or the insulin analogue is administered to the subject in the amount of 0.1 to 2 units/kg/day.

6. The method of claim 1, wherein the (R)-(+)-verapamil or a pharmaceutically acceptable salt thereof is administered to the subject in single or divided doses 2 or 3 times each day; and the insulin or the insulin analogue is administered to the subject in single or divided doses 2 or 3 times each day.

7. A pharmaceutical composition for the treatment of diabetes mellitus and/or disorders related to diabetes mellitus comprising:
(R)-(+)-verapamil or a pharmaceutically acceptable salt thereof;
insulin or an insulin analogue; and
a pharmaceutically acceptable excipient.

8. The pharmaceutical composition of claim 7, wherein the insulin analogue is glargine, degludec or detemir.

9. The pharmaceutical composition of claim 8, wherein the insulin analogue is glargine.

10. (canceled)
